(19) United States
(12) Patent Application Publication
(10) Pub. No.: US 2025/0381378 A1
(43) Pub. Date: Dec. 18, 2025

(54) TATTOO ASSEMBLY WITH VISUAL ENHANCEMENT

(71) Applicant: Jean Felix Lacroix, Boca Raton, FL (US)

(72) Inventor: Jean Felix Lacroix, Boca Raton, FL (US)

(21) Appl. No.: 19/241,010

(22) Filed: Jun. 17, 2025

Related U.S. Application Data
(60) Provisional application No. 63/660,782, filed on Jun. 17, 2024.

(51) Int. Cl. A61M 37/00; H04N 23/63

(52) U.S. Cl. CPC A61M 37/0076; H04N 23/63

(57) ABSTRACT

A tattoo assembly with visual enhancement that includes a handheld body defining a needle opening for a needle member to electrically reciprocate therethrough so as to tattoo or otherwise penetrate a user's skin to leave permanent markings with indelible ink, that includes a camera coupled to the handheld body, with a lens configured to be oriented toward the second end of the handheld body to capture at least one enhanced image of a distal terminal end of the needle member, and that includes an electronic display communicatively coupled to the camera, coupled to the handheld body, and operably configured to display the at least one enhanced image of the distal terminal end of the needle member thereon for viewing by the user.

### FIELD OF THE INVENTION

The present invention relates generally to tattooing machines and, more particularly, relates to tattoo machines having a camera and a display.

### BACKGROUND OF THE INVENTION

A tattoo is a form of body modification made by inserting tattoo ink, dyes, and/or pigments, either indelible or temporary, into the dermis layer of the skin to form a design. There are many different types of tattoo machines and processes utilized by users to create tattoos. When creating a tattoo, precision and accuracy are paramount to many users and recipients, particularly when creating tattoos with a significant amount of detail. In many instances, however, users have a difficult time either viewing the exact location of the needle when entering the user's skin and/or want to ensure the precise location of the needle when entering the user's skin. Some known tattoo machines are ergonomically shaped to enable a user to view the distal end of the needle, but these known machines can be difficult or uncomfortable to handle and still do not provide users the ability to closely view the target area for the needle.

Another known tattoo device includes a tattoo machine with attached camera, as disclosed in Smith, U.S. Pat. No. 10,525,245. This tattoo machine includes a camera mount for coupling the camera assembly to the tattoo machine, wherein the camera mount may be attached to an adjustable arm. This assembly fails to provide users the ability for the user to view the target area in real time or on an electronic display. Furthermore, the flexible arm in which the camera mount is installed is prone to vibration and the overall assembly fails to provide users the ability enhance or control the transmission of the images in an effective, effective, and safe manner.

Therefore, a need exists to overcome the problems with the prior art as discussed above.

### SUMMARY OF THE INVENTION

The invention provides a tattoo assembly with visual enhancement that overcomes the hereinafore-mentioned disadvantages of the heretofore-known devices and methods of this general type and that provides users real-time enhanced video of a target area that is being tattooed in an effective, efficient, and safe manner.

With the foregoing and other objects in view, there is provided, in accordance with the invention, a tattoo assembly with visual enhancement that includes a handheld body with an outer surface, with a first end, with a second end opposing the first end and defining a needle opening, and with a needle member movably retained within the handheld body and electrically and configured to reciprocate through the needle opening. The assembly also includes a camera coupled to the handheld body, with a lens configured to be oriented toward the second end of the handheld body to capture at least one enhanced image of a distal terminal end of the needle member and an electronic display communicatively coupled to the camera, coupled to the handheld body, and operably configured to display the at least one enhanced image of the distal terminal end of the needle member thereon.

In accordance with another feature, an embodiment of the present invention includes an electromagnet disposed within the handheld body and electrically coupled to a switch operably configured to electrically complete a circuit between the electromagnet and a power source. Furthermore, a battery may form the power source and including a metallic prong operably configured to be inserted into an electrical port electrically coupled to the switch and the electrical port may be defined on the first end of the handheld body.

In accordance with a further feature of the present invention, the needle member is operably configured to electromagnetically reciprocate.

In accordance with yet another feature of the present invention, the at least one enhanced image includes at least a 2× magnification.

In accordance with an exemplary feature of the present invention, the camera is operably configured to capture a plurality of enhanced images forming a video of the reciprocating movement of the distal terminal end of the needle member.

In accordance with a further feature of the present invention, the electronic display is operably configured to display the video of the reciprocating movement of the distal terminal end of the needle member thereon.

In accordance with another feature, an embodiment of the present invention also includes at least one LED coupled to the handheld body and configured to be oriented toward the second end of the handheld body.

In accordance with yet another feature, an embodiment of the present invention also includes an electronic controller communicatively coupled to the camera and the electronic display and operably configured to modulate electromechanical settings on at least one of the camera and the electronic display.

In accordance with an additional feature, an embodiment of the present invention also includes a display extension member telescopically coupled to the electronic display and the handheld body, wherein the display extension member is operably configured to be extended and retracted, and the electronic display is rotatably coupled to the display extension member.

In accordance with another feature, an embodiment of the present invention includes a camera extension member coupled with an and the handheld body and coupled to the camera in a cantilevered configuration to offset the lens of the camera a length of at least 2 cm above the outer surface of the handheld body.

In accordance with a further feature of the present invention, the camera is rotatably coupled to the camera extension member and camera and the electronic display are coupled to the handheld body with an annular clasp and fastener. The fastener may be one or both a set screw and/or a magnet.

In accordance with an additional feature of the present invention, the camera and the electronic display are each respectively and independently coupled to the handheld body with an annular clasp and fastener.

Also in accordance with the present invention, a tattoo assembly with visual enhancement is disclosed that includes a handheld body with an outer surface, with a switch, defining a needle opening, with an electromagnet coupled thereto and electrically coupled to the switch that is operably configured to electrically complete a circuit between the electromagnet and a battery, and with a needle member movably retained within handheld body and that is electrically and operably configured to reciprocate through the needle opening utilizing the electromagnet when the circuit is complete, a camera coupled to the handheld body, with a lens configured to be oriented toward the second end of the handheld body to capture an at least 2× magnification image of an area proximal to the needle opening, and an electronic display communicatively coupled to the camera, electrically coupled to the battery, coupled to the handheld body, and operably configured to display the at least one 2× magnification of the area proximal to the needle opening.

Although the invention is illustrated and described herein as embodied in a tattoo assembly with visual enhancement, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not necessarily drawn to scale but, where applicable, may be utilized to support a particular structural configuration or geometric relationship between components utilized in the assembly.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time. Also, for purposes of description herein, the terms “upper”, “lower”, “left,” “rear,” “right,” “front,” “vertical,” “horizontal,” and derivatives thereof relate to the invention as oriented in the figures and is not to be construed as limiting any feature to be a particular orientation, as said orientation may be changed based on the user's perspective of the device. Furthermore, there is no intention to be bound by any expressed or implied theory presented in the preceding technical field, background, brief summary or the following detailed description.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. In this document, the term “longitudinal” should be understood to mean in a direction corresponding to an elongated direction of the needle member or the direction in which the needle member reciprocates. The terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a perspective view of a tattoo assembly with visual enhancement in accordance with one embodiment of the present invention;

FIG. 2 is a perspective view of the tattoo assembly in FIG. 1;

FIG. 3 is a partially exploded view of the tattoo assembly in FIG. 1;

FIGS. 4-5 are additional perspective views of the tattoo assembly in FIG. 1;

FIG. 6 is a cross-sectional view of a representative tattoo assembly with visual enhancement in accordance with one embodiment of the present invention;

FIG. 7 is a block diagram depicting exemplary components utilized in a tattoo assembly with visual enhancement in accordance with one embodiment of the present invention;

FIG. 8 is a perspective view of a tattoo assembly with visual enhancement in accordance with one embodiment of the present invention;

FIG. 9 is an exploded view of the tattoo assembly in FIG. 8;

FIG. 10 is a perspective view of the tattoo assembly in FIG. 8;

FIG. 11 is a top plan view of the tattoo assembly in FIG. 8;

FIG. 12 is a perspective view of a tattoo assembly with visual enhancement in accordance with one embodiment of the present invention;

FIG. 13 is an exploded view of the tattoo assembly in FIG. 8;

FIG. 14 is a perspective view of the tattoo assembly in FIG. 8; and

FIG. 15 is a top plan view of the tattoo assembly in FIG. 8.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

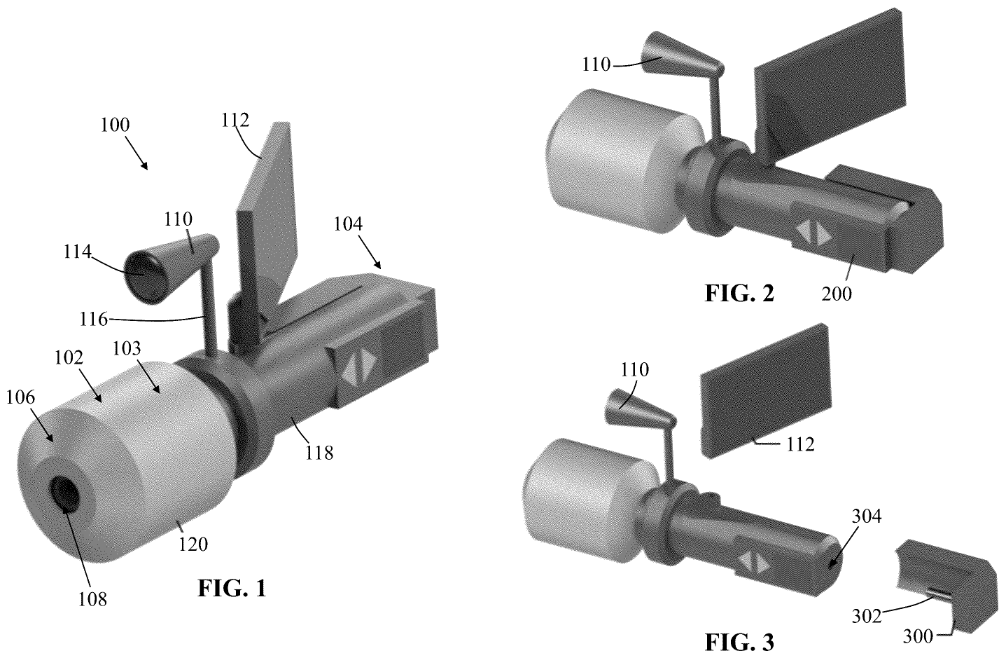

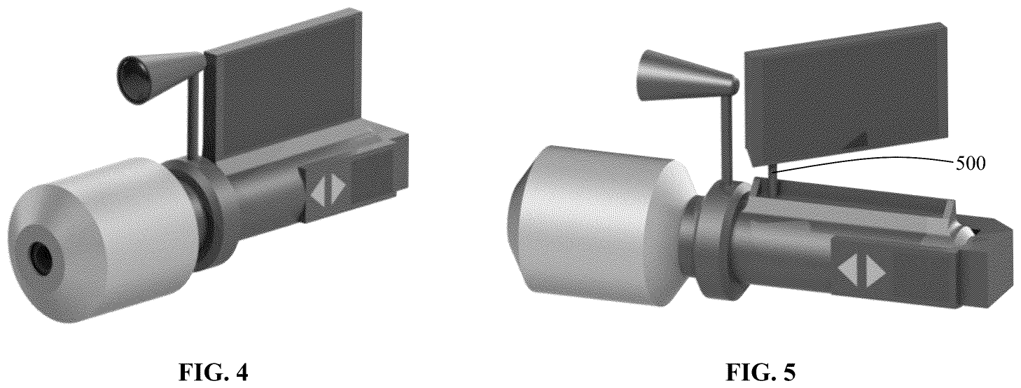

The present invention provides a novel and efficient tattoo assembly with visual enhancement that enables users to view the target area on the person's skin the user desires to tattoo. Referring now to FIG. 1, one embodiment of the present invention is shown in a perspective view. The figures depicted herein show several advantageous features of the present invention, but, as will be described below, the invention can be provided in several shapes, sizes, combinations of features and components, and varying numbers and functions of the components. The first example of a tattoo assembly with visual enhancement as shown in FIG. 1 includes a tattoo assembly 100 with visual enhancement that includes a handheld body 102, a camera 110 configured to capture the area in which the user desires to tattoo, and an electronic display 112 configured to display the captured area for the user to view. Beneficially, the camera 110 utilized to capture the area is configured to capture enhanced image(s) and/or video, wherein enhanced image(s) and/or video may include high resolution and/or magnified image(s) and/or video of the target area.

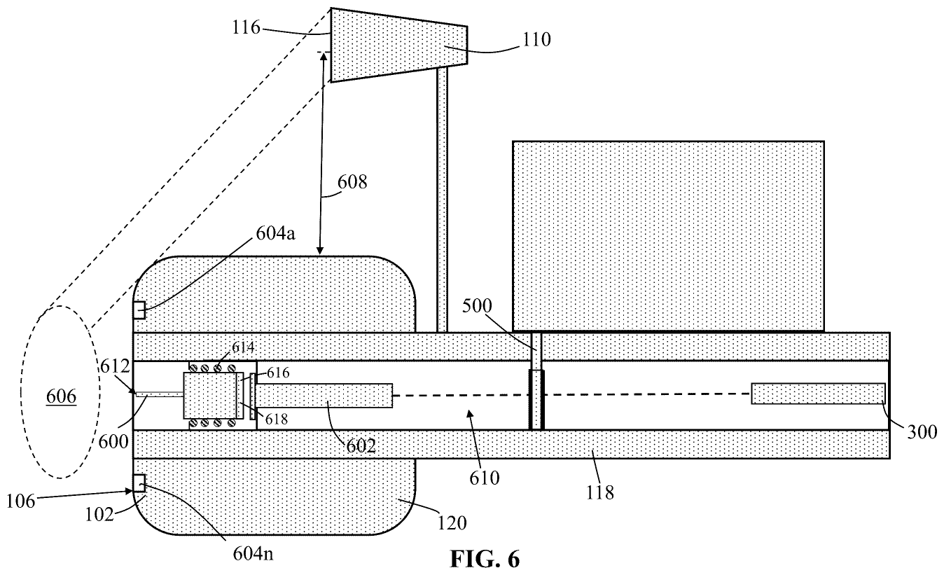

The handheld body 102 may include a sidewall 118 defining a cavity 610 (best seen in FIG. 6) for housing and shielding components utilized by the assembly 100 to operate. As used herein, the term “wall” is intended broadly to encompass continuous structures, as well as, separate structures that are coupled together so as to form a substantially continuous external surface. The body 102 may be made of a lightweight metallic material, e.g., stainless steel or aluminum, or a polymeric material that is preferably heat resistant. The body 102 may be shaped and sized to be handheld, or capable and configured to be held by a user's single hand. To that end, the body 102 may have an average maximum diameter or length along its full longitudinal length that is less than approximately 6 in. The body 102 may include one or more grip attachments, e.g., attachment 120, ergonomically shaped, textured, and/or configured to be grasped by the user safely and effectively. In one embodiment, the attachment 120 may be fastened to the sidewall 118 and may be cylindrical in shape.

The handheld body 102 includes an outer surface 103, a first end 104, and a second end 106 opposing the first end 104 and defining a needle opening 108 that may be approximately 0.25-lin. The attachment 120 may also include an opening configured to be aligned with the needle opening 108, wherein the openings may be preferably enclosed. With reference to FIG. 1 and FIG. 6, the assembly 100 also includes a needle member 600 that is substantially rigid and movably retained within the handheld body 102. The needle member 600 is electrically and/or magnetically operated, and configured to reciprocate through the needle opening 108 to puncture the skin of an individual. In some embodiments, the needle member 600 reciprocates through the needle opening 108 at approximately 0.8 to 50 cycles per second. Furthermore, either or both the distal end 612 of the needle member 600 and a portion of a length of the needle member 600 reciprocate through the opening 108 so as to tattoo or otherwise penetrate a person's skin to leave permanent markings with indelible ink exposed on the distal end 612 of the needle member 600. To that end, the distal end 612 of the needle member 600 may be tapered or sharp.

Now referring to FIG. 1 and FIGS. 6-7, in one embodiment, the handheld body 102 has a total longitudinal length of approximately 5-10 in. separating the first and second ends 104, 106 and utilizes an electromagnet 602, that may be of coil configuration, disposed within the handheld body 102 to operate the needle member 600. More specifically, the electromagnet 602 may be electrically coupled to a switch 700 that is operably configured to electrically complete a circuit between the electromagnet 700 and a power source, e.g., a battery 300 or electrical wall outlet. As such, the needle member 600 may be operably configured to electromagnetically reciprocate back-and-forth through the opening 108. Preferably, the assembly 100 utilizes a battery 300 so it can be more effectively handheld.

With reference to FIGS. 2-3 and FIG. 7, the battery 300 forms the power source on the assembly 100 and may include a metallic prong 302 operably configured to be inserted into an electrical port 304 electrically coupled to the switch 700. To provide a balanced assembly 100, the electrical port 304 is defined on the first end 104 of the handheld body 102 so the battery 300 can be selectively removed and inserted. The battery 300 may include a body housing the cells that is shaped and sized to contour the outer surface 103 of the housing 102 and may be lithium-ion battery. The outer surface 103 may include a haptic control or power indicator screen 200 that may visually depict the remaining battery percentage of the battery 300. The screen 200 may also enable user control of the magnification of camera 110.

The camera 110 is preferably rotatably coupled to the handheld body 102 so the user can angle or orient the lens 116 of the camera 110 toward the target area 606. Said another way, the lens 116 of the camera 110 is configured to be oriented 112 toward the second end 106 of the handheld body 102 to capture one or more enhanced image(s) of the distal terminal end 612 of the needle member 600. Stated differently, the lens 116 of the camera 110 is configured to be oriented toward the second end 106 of the handheld body 102 to capture one or more at least 2× magnification of the target area 606 proximal to the needle opening 108 (i.e., at or near, within 2 in of the target's skin area 606). The target area 606 is represented in FIG. 6, but may not be necessarily circular. The lines indicated in FIG. 6 represent a line-of-sight for the camera 110 so that the user can effectively see the desired area. In one embodiment, the camera 110 may be USB digital microscopic magnification camera.

In one embodiment, the at least one enhanced image includes at least a 2× magnification or greater image of the target area 606 and/or distal end 612 of the needle member 600 when exiting the assembly 100. For example, in one embodiment, the magnification may be a 3× digital zoom, a 4.5-180× zoom, a 450-510× zoom, a 15-60× zoom, a 18-720× zoom, a 1800-2040× zoom, or a 60-240× zoom that may be output onto the electronic display 112. Preferably, the camera 110 is operably configured to capture a plurality of enhanced images forming a video of the reciprocating movement of the distal terminal end 612 of the needle member 600 and/or the target area 606. In preferred embodiments, the camera 110 includes software configured to identify and track a target structure or area (e.g., area 606) and/or providing for screen stabilization software in combination with the gimbal of the assembly 100.

The assembly 100 also includes electronic display 112 that may of a liquid-crystal display (LCD) communicatively coupled to the camera 110, coupled to the handheld body 102, and operably configured to display the one or more enhanced image(s) of the distal terminal end 612 of the needle member 600 thereon, preferably in real time so the user may make modification(s) or adjustment(s). The electronic display 112 is preferably an ultra-high definition 2160P display for effective viewing of the target area 606 and/or distal end 612. Furthermore, the electronic display 112 is operably configured to display the video of the reciprocating movement of the distal terminal end 612 of the needle member 600 thereon.

The camera 110 and display 112 may be preferably independent of one another structurally to provide for independent control and movement. Furthermore, the camera 110 and display 112 may be rotatably, extendably, and retractably coupled to the body 102 using one or more extension members that are preferably substantially rigid, e.g., made with a hard polymeric or metallic material. For example referencing FIG. 1 and FIG. 5, a display extension member 500 may be utilized and that is rotatably and telescopically coupled to the electronic display 112 and the handheld body 102 allowing for selective control and storage. The display extension member 500 is operably configured to be extended and retracted, and the electronic display 112 may be rotatably coupled to the display extension member 500. A camera extension member 114 may also be utilized and is coupled to the handheld body 102 and coupled to the camera 110 in a cantilevered configuration. In one embodiment, as best seen in FIG. 6, the camera extension member 114 is configured to offset the lens 114 of the camera 110 a length 608 of at least 2 cm (or approximately 1 in) above the outer surface 104 of the handheld body 102.

In one embodiment, the camera 110 is rotatably coupled and telescopically coupled to the camera extension member 114. Rotatably coupled may include pivoting and approximately 360° rotation. In one embodiments, the camera 110 and display 112 may be coupled to an extension member enabling rotation on one or more bearing(s) and may be frictionally engaged such that said camera 110 and display 112 are secured on the respective extension member until an external force is applied, e.g., 1-2 lbf. Each of the extension members may be elongated and may include sections configured to telescopic extension and retraction (within the handheld body 102).

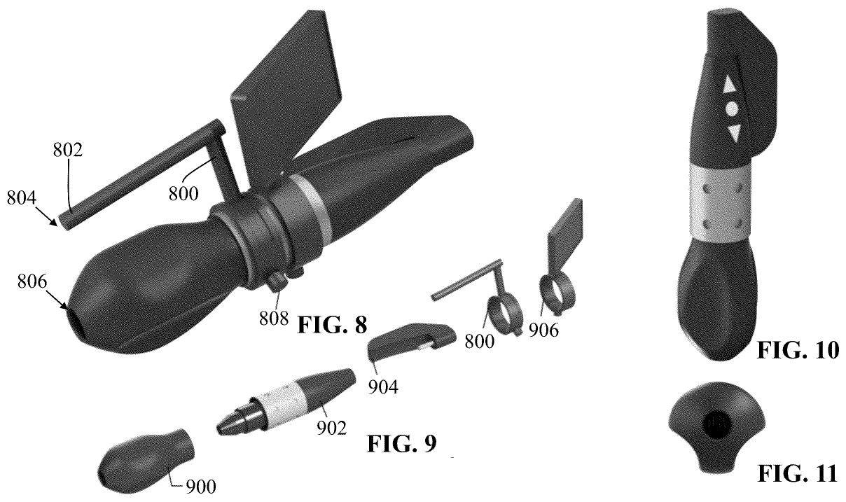

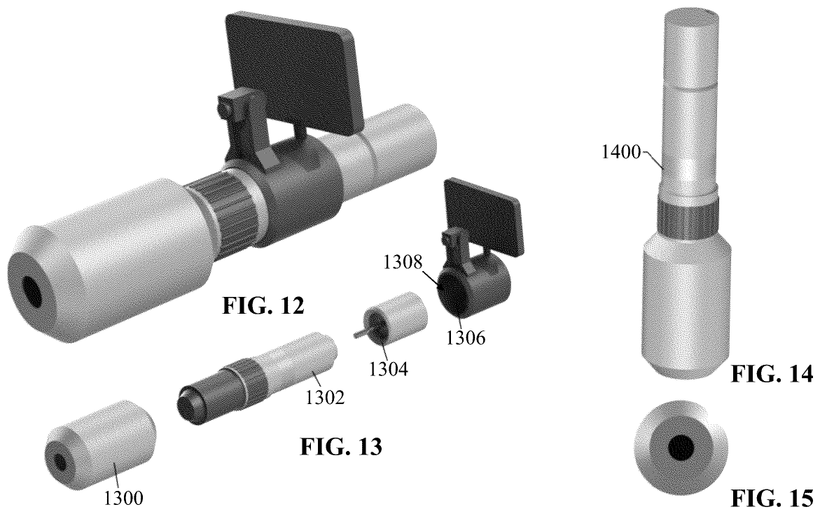

In one embodiment, as best shown in FIGS. 8-9, the camera extension member 800 offsets the camera 802 and the lens 804 is configured by the camera body to be disposed above and proximal (as defined herein) to the second end 806. FIGS. 8-9 also depict an assembly having a grip 900, a body 902 to which the grip 900 is attached, a selectively removable battery 904, a camera 906, and an electronic display 908. Similarly, FIGS. 12-15 also depict an assembly having a grip 1300, a body 1302 to which the grip 1300 is attached, a selectively removable battery 1304, and a camera and electronic display body 1306. The camera and the electronic display may be coupled to the handheld body with an annular clasp and fastener (e.g., a set screw 808 and/or magnet 1400), as depicted in FIGS. 8-15. In other embodiments, the camera and the electronic display may be coupled to the handheld body with a fastener. As seen depicted in FIGS. 8-11, the body 902 may include a plurality of threaded apertures disposed around the circumference of the body 902 for receiving the set screw. The extension member 800 may be operable to rotate within the annular clasp to a desired angle and the there may be two rows of circumferentially disposed threaded apertures (approximately 0.25 in apart from one another) for retaining both a clasp member for the camera and the electronic display. Similarly, the magnetic portion 1400 of the body 1302 may surround the body 1302 and for receiving a magnetic portion 1308 of the camera body and/or display body. In one embodiment, the camera and the electronic display are each respectively and independently coupled to the handheld body with an annular clasp and fastener, as best depicted in FIG. 8. In some embodiments, however, the camera and display may be combined in a unitary and monolithic body.

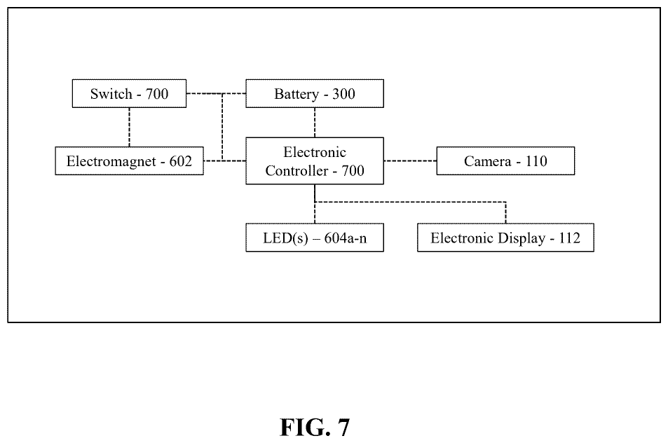

FIG. 7 depicts exemplary communication and/or electrical connections between the components utilized in the assembly, including the display 112 and the camera 110. These connections, which are represented with dashed lines, may be wired or wireless connections. In one embodiment, the assembly 100 may include one or more LED(s) 604a-n coupled to the handheld body 102, wherein “n” represents any number greater than one. The LED(s) 604a-n may be configured to be oriented 112 toward the second end 106 of the handheld body 102 so as to provide light to visually perceive and capture the at least one enhanced image of a distal terminal end of the needle member 600 or the target's skin area 606 in which the needle member 600 penetrates.

In preferred embodiments of the present invention, the assembly includes an electronic controller 700 that is communicatively coupled to the camera 110, electronic display 112, and other electrical and/or mechanical components of the assembly 100. The controller 700 may be operably configured to modulate electromechanical settings on at least one of the camera 110 and the electronic display 112, e.g., through the control panel or buttons 200. The electronic controller 700 may be configured to dynamically alter the field produced by the electromagnetic coil through machine learning. By way of example and not limitation, the control unit 700 may compare the intended acceleration of the needle during a needle cycle period to the actual measured acceleration of the needle to determine the resistance of the target skin. As seen in FIG. 6, the needle member 600 is attached to a needle slide having springs 614 biasing the slide, which is in turn attached to a permanent magnet 616 retained with an o-ring 618. The needle member 600 is thus configured to reciprocate within the body 102 by activation of the electromagnetic coil 602 generating an oscillating magnetic field.

Although a specific order of executing utilization or set-up steps has been disclosed, the order of executing the steps may be changed relative to the order shown in certain embodiments. Also, two or more steps described or shown as occurring in succession may be executed concurrently or with partial concurrence in some embodiments. Certain steps may also be omitted for the sake of brevity. In some embodiments, some or all of the process steps can be combined into a single process.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the above described features.

What is claimed is:

1. A tattoo assembly with visual enhancement comprising:
a handheld body with an outer surface, with a first end, with a second end opposing the first end and defining a needle opening, and with a needle member movably retained within the handheld body and electrically and configured to reciprocate through the needle opening;
a camera coupled to the handheld body, with a lens configured to be oriented toward the second end of the handheld body to capture at least one enhanced image of a distal terminal end of the needle member; and
an electronic display communicatively coupled to the camera, coupled to the handheld body, and operably configured to display the at least one enhanced image of the distal terminal end of the needle member thereon.

2. The tattoo assembly with visual enhancement according to claim 1, further comprising:
an electromagnet disposed within the handheld body and electrically coupled to a switch operably configured to electrically complete a circuit between the electromagnet and a power source.

3. The tattoo assembly with visual enhancement according to claim 2, further comprising:
a battery forming the power source and including a metallic prong operably configured to be inserted into an electrical port electrically coupled to the switch.

4. The tattoo assembly with visual enhancement according to claim 3, wherein the electrical port is defined on the first end of the handheld body.

5. The tattoo assembly with visual enhancement according to claim 1, wherein the needle member is operably configured to electromagnetically reciprocate.

6. The tattoo assembly with visual enhancement according to claim 1, wherein the at least one enhanced image includes at least a 2× magnification.

7. The tattoo assembly with visual enhancement according to claim 1, wherein the camera is operably configured to capture a plurality of enhanced images forming a video of the reciprocating movement of the distal terminal end of the needle member.

8. The tattoo assembly with visual enhancement according to claim 7, wherein the electronic display is operably configured to display the video of the reciprocating movement of the distal terminal end of the needle member thereon.

9. The tattoo assembly with visual enhancement according to claim 1, further comprising:
at least one LED coupled to the handheld body and configured to be oriented toward the second end of the handheld body.

10. The tattoo assembly with visual enhancement according to claim 1, further comprising:
an electronic controller communicatively coupled to the camera and the electronic display and operably configured to modulate electromechanical settings on at least one of the camera and the electronic display.

11. The tattoo assembly with visual enhancement according to claim 1, further comprising:
a display extension member telescopically coupled to the electronic display and the handheld body, wherein the display extension member is operably configured to be extended and retracted and the electronic display is rotatably coupled to the display extension member.

12. The tattoo assembly with visual enhancement according to claim 1, further comprising:
a camera extension member coupled with an and the handheld body and coupled to the camera in a cantilevered configuration to offset the lens of the camera a length of at least 2 cm above the outer surface of the handheld body.

13. The tattoo assembly with visual enhancement according to claim 1, wherein the camera is rotatably coupled to the camera extension member.

14. The tattoo assembly with visual enhancement according to claim 1, wherein the camera and the electronic display are coupled to the handheld body with an annular clasp and fastener.

15. The tattoo assembly with visual enhancement according to claim 14, wherein the fastener is at least one of a set screw and a magnet.

16. The tattoo assembly with visual enhancement according to claim 1, wherein the camera and the electronic display are each respectively and independently coupled to the handheld body with an annular clasp and fastener.

17. A tattoo assembly with visual enhancement comprising:
a handheld body with an outer surface, with a switch, defining a needle opening, with an electromagnet coupled thereto and electrically coupled to the switch that is operably configured to electrically complete a circuit between the electromagnet and a battery, and with a needle member movably retained within handheld body and that is electrically and operably configured to reciprocate through the needle opening utilizing the electromagnet when the circuit is complete;
a camera coupled to the handheld body, with a lens configured to be oriented toward the second end of the handheld body to capture an at least 2× magnification image of an area proximal to the needle opening; and
an electronic display communicatively coupled to the camera, electrically coupled to the battery, coupled to the handheld body, and operably configured to display the at least one 2× magnification of the area proximal to the needle opening.

18. The tattoo assembly with visual enhancement according to claim 14, wherein the handheld body further comprises:
a first end and a second end opposing the first end and defining the needle opening, wherein the lens is configured to be oriented toward the second end of the handheld body to capture the at least one 2× magnification.

19. The tattoo assembly with visual enhancement according to claim 14, further comprising:
an electronic controller communicatively coupled to the camera and the electronic display and operably configured to modulate electromechanical settings on at least one of the camera and the electronic display.

20. The tattoo assembly with visual enhancement according to claim 14, further comprising:
a camera extension member coupled with an and the handheld body and coupled to the camera in a cantilevered configuration to offset the lens of the camera a length of at least 2 cm above the outer surface of the handheld body.
